(19) United States
(12) Patent Application Publication
(10) Pub. No.: US 2023/0144089 A1
(43) Pub. Date: May 11, 2023

(54) SMART SYSTEM FOR SKIN TESTING AND CUSTOMISED FORMULATION AND MANUFACTURING OF COSMETICS

(71) Applicant: ABBI SAS, Sainte-Foy-Les Lyon (FR)

(72) Inventors: Joanna IFERGAN, DARDILLY (FR); Frédéric IFERGAN, DARDILLY (FR)

(21) Appl. No.: 17/918,208

(22) PCT Filed: May 19, 2021
(86) PCT No.: PCT/IB2021/054329

§ 371 Date: Oct. 11, 2022

(30) Foreign Application Priority Data
May 20, 2020 (FR) FR2005259

(51) Int. Cl. G06T 7/00; G06V 40/16; A45D 44/00

(52) U.S. Cl. CPC G06T 7/0016; A45D 44/005; G06V 40/161; G06V 40/168; A45D 2044/007; G06T 2207/30088; G06T 2207/30201

(57) ABSTRACT

A process to identify a tailor-made cosmetic formula adapted to an user, including a step of collecting picture or video information, a step of storing said information into a database, a step of storing said information into a database, a step of comparing the collected information with the information in the database, a step of devising analysis results, characterized in that said process comprises a step of submission of a cosmetic formulation including at least one cosmetic base and/or at least one active ingredient tailored to the user.

The invention relates to a process to figure out a tailor-made formula adapted to an user in the cosmetic area. In particular, the invention relates to an examination of the skin that allows the identification of the tailor-made cosmetic formulation including at least a cosmetic base and at least one active ingredient. This kind of examination can be implemented, for example, by an smart mirror.

“Tailor-made formation” means any kind of combination or compound of active ingredients and bases that may confer final specific properties in the area of cosmetics. These combinations are valid for a given person; taking into account any kind of parameter required to assess a result of the formulation. For example, a parameter could be a feature of the skin.

“Cosmetic base” means a substance or a compound designed for contact with various outer parts of the skin, in particular the epidermis. The base of a cosmetic product is all that makes it except the pure active ingredients. It can be in the form of an emulsion, a cream, a gel, a creamy gel, a balm, a serum, an oil or a combination of the same. It can admit pure active ingredients. It may have various textures according to the type of skin and often accounts for more than 80% of the total volume of a cosmetic product.

“Active ingredient” means a highly concentrated substance, thus introduced in very small amount into a cosmetic product. It is also the active part of the cosmetic product, which means it provides some action and confers some properties to the cosmetic product. This makes it possible to identify the nature of the cosmetic product and aim at the identified condition. Active ingredients are in relation with the types of skins and to the skin damages.

“Smart mirror” is a device capable of implementing several functions. In particular, a smart mirror can take a photograph, record information into a database and provide results of an analysis as a display on screen.

When using cosmetic products, users may use a product that is not adapted to their skin, i.e. that may induce adverse effects. The user may also use a product that does not bring the expected results, i.e. a product with little efficiency or with weak and slow progression. Thus, there is a need in this domain to develop tailor-made formulations that are adapted to any specific user.

It is known from the prior art the solution provided by the patent US9760935B2 about a process to advocate products and beauty treatments, skin care and anti-ageing products according to the analysis of only one or several photographs or videos of a user.

However, the solution from this document is mainly predictive. It is designed to provide the user a prediction relying on a recommendation of products from statistical results from several other users. Thus, it does not provide a tailor-made solution to the user.

It is known from the prior art the solution provided by the patent L1S2019Q026013A1 about a process implemented in a computer appliance with a display screen and a processor to provide an improvement interface for cosmetic products from a digital photograph including a plurality of facial characteristics.

However, this solution is designed to only select and advocate an existing cosmetic product that matches a selected facial characteristic and provide a prediction of the face by image display and image processing. Here again, the solution from this document cannot advocate a new cosmetic formulation that is tailored to a specific user.

Our invention is designed to overcome the problems in the state of art thanks to a skin examination process that provides a tailor-made cosmetic formulation and produces a tailor-made cosmetic product to an user.

The invention consists in a process to identify a tailor-made cosmetic formula adapted to an user, including a step of collecting picture or video information, a step of storing said information into a database, a step of storing said information into a database, a step of comparing the collected information with the information in the database, a step of devising analysis results, characterized in that said process comprises a step of submission of a cosmetic formulation including at least one cosmetic base and/or at least one active ingredient tailored to the user.

According to an embodiment, the process is characterized in that it includes a system of sensors capable of detecting the face of an user and preferably at least one condition selected among roughness, cracks of the .skin, sebum excess, .scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

According to an embodiment, the process is characterized in that at least one active ingredient is identified for at least one of the conditions selected among roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

According to an embodiment, the process is characterized in that a scan of the face is made on at least 70% of the contour of the face, including benchmarks selected among the eyes, the nostrils or the lips.

According to an embodiment, the process is characterized in that the scan on a full face is at least 99.8%.

According to an embodiment, the process is characterized in that the scan on a full face with a marked zone of the skin is at least 98%.

According to an embodiment, the process is characterized in that a cosmetic base is selected among a cream, a serum, an oil, a balm, a gel, a creamy gel, an emulsion or a combination of the same.

According to an embodiment, the process is characterized in that figuring out a tailor-made cosmetic formulation includes the following steps:
A step of taking a photograph and detecting the face in the photograph.
A step of delineation of the contours of the face for analysis and division of the face in zones.
A step of analysis of the skin from said photograph.
A step of rating from the analysis.
A step of creation of a tailor-made cosmetic formulation.
A step of creation of a tailor-made cosmetic product from said tailor-made cosmetic formulation.
A step of comparative analysis of a result before and after application of the cosmetic product.

The invention also includes a smart mirror device capable of implementing the process.

Other features and advantages of the process will appear with the description below and appended figures.

However, this solution is designed to only select and advocate an existing cosmetic ,product that matches a selected facial characteristic and provide a prediction of the face by image display and image processing. Here again, the solution from this document cannot advocate a new cosmetic formulation that is tailored to a specific user.

Our invention is designed to overcome the problems in the state of art thanks to a skin examination process that provides a tailor-made cosmetic formulation and produces a tailor-made cosmetic product to an user.

The invention consists in a process to identify a tailor-made cosmetic formula adapted to an user, including a step of collecting picture or video information, a step of storing said information into a database, a step of storing said information into a database, a step of comparing the collected information with the information in the database, a step of devising analysis results, characterized in that said process comprises a step of submission of a cosmetic formulation including at least one cosmetic base and/or at least one active ingredient tailored to the user.

According to an embodiment, the process is characterized in that it includes a system of sensors capable of detecting the face of an user and preferably at least one condition selected among roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

According to an embodiment, the process is characterized in that at least one active ingredient is identified for at least one of the conditions selected among roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

According to an embodiment, the process is characterized in that a scan of the face is made on at least 70% of the contour of the face, including benchmarks selected among the eyes, the nostrils or the lips.

According to an embodiment, the process is characterized in that the scan on a full face is at least 99.8%.

According to an embodiment, the process is characterized in that the scan on a full face with a marked zone of the skin is at least 98%.

According to an embodiment, the process is characterized in that a cosmetic base is selected among a cream, a serum, an oil, a balm, a gel, a creamy gel, an emulsion or a combination of the same.

According to an embodiment, the process is characterized in that figuring out a tailor-made cosmetic formulation includes the following steps:
A step of taking a photograph and detecting the face in the photograph.
A step of delineation of the contours of the face for analysis and division of the face in zones.
A step of analysis of the skin from said photograph.
A step of rating from the analysis.
A step of creation of a tailor-made cosmetic formulation.
A step of creation of a tailor-made cosmetic product from said tailor-made cosmetic formulation.
A step of comparative analysis of a result before and after application of the cosmetic product.

The invention also includes a smart mirror device capable of implementing the process.

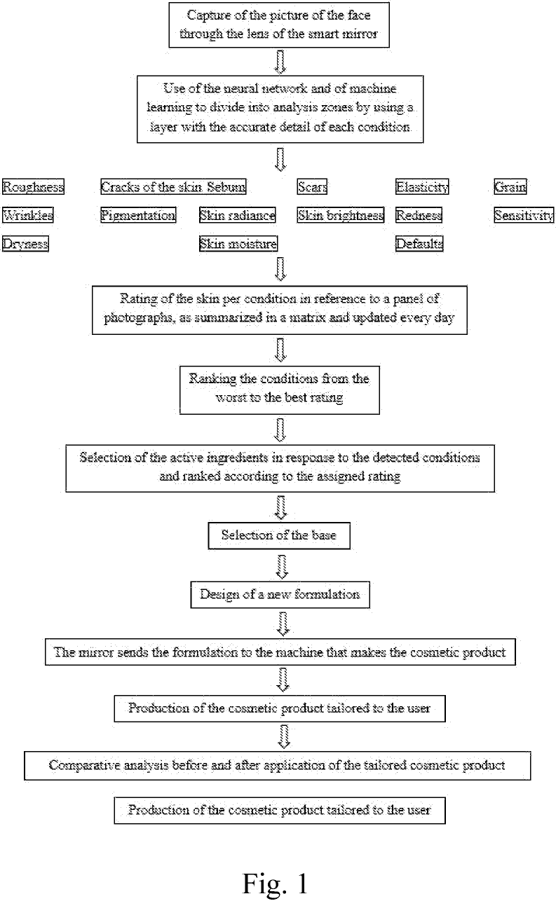

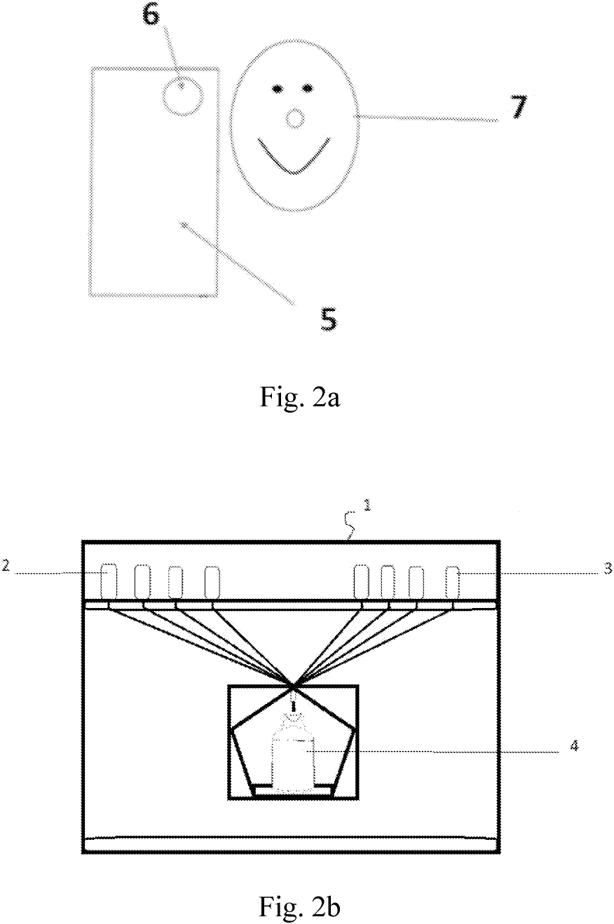

Other features and advantages of the process will appear with the description below and appended figures.

[FIG. 1] The FIG. 1 shows a recapitulative diagram of the main steps of the process.

[FIG. 2a] The FIG. 2a shows an appliance according to the invention to detect the face of an user.

[FIG. 2b] The FIG. 2b shows an appliance according to the invention to produce a tailor-made cosmetic product.

The FIG. 1 resumes the main steps of the process.

The first step consists in the user taking a photograph, then detecting the face in the photograph.

There is no need for the user to pose in any specific way. A simple front snapshot around the face is enough. The user can get it with an appliance, such as a smart mirror or any means including a system to capture and display a picture.

The scan of the face of an user relies on an artificial neural network and/or “machine learning” trained on more than 50.000 faces. The scan on a full face is at least 99.9%. The scan is at least 98% on colored skins and very marked faces. The scan also works on a face with reduced contour, which means a face with an inner contour less than 100% down to 70%.

The second step consists in a delineation of the contours of the face for analysis and division in zones.

According to an embodiment of the process, the second step is to delinate the accurate contour to exclude, for example, the background of the picture, the neck and the hair. To do so, artificial intelligence relies on 2 pillars:
The scan of benchmarks that may be, for example, the eyes, the nostrils and the lips.
A network of specific artificial neurons to detect blur contours.

The result advantageously consists in a set of abutting geometric shapes that can tie more than 100 dots on the face and can both identify each zone to be specifically treated and the zones off treatment.

For example, a comparison between the rhombus between the eye and the ear, on one hand, and the rhombus between the forehead and the cheekbone, on the other hand, shows a necessarily different illumination of the nose. Thus, possible discrepancies in connection with the same specific zone will be advantageously better taken into account.

Regarding zones off treatment, for example, the process systematically excludes the eye, the mouth and the nostrils.

In addition, the process takes into account another kind of zone off treatment, i.e. the detection of “non-skin” zones by the artificial intelligence. For example, this is a set of algorithms that scan the defined .analysis zones to detect exceptions, such as glasses, ,piercings, possible wounds or aberrations resulting from wrong illumination (for example, the effect of extreme glint or too strong shades).

Excluding such zones off treatment advantageously allows better focus on the zones of interest, hence a better detection of the accurate contours of the face.

In particular, said algorithms start by learning colour and lighting characteristics of each zone to calculate an average, then to deduce relative exceptions.

For example, too strong a shade is excluded from a too marked skin, while the same colour with the same level of lighting may be included on a dark-skinned face.

Specific algorithms have been developed for each analysis. Indeed, as a rule, they can adapt according to the general colour of the skin. They can take into account the specificity of each zone and may exclude extreme values too far from the calculated average value.

Thus, these algorithms are advantageously auto-adaptative.

It was not possible to define the rules and objects to detect for a few conditions to analyze; thus, a machine learning system has been designed. Indeed, a neural network and/or “machine learning” dedicated to each analysis involved is trained on a minimum 2500-picture database of the object to detect.

The third step consists in an analysis of the skin from the photograph. The main objects to analyse relate to skin conditions such as roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

For wrinkles, the artificial intelligence shall detect variations in colour that make it possible to define a wrinkle by shape and position.

Advantageously, the visual delineation of each wrinkle is pictured in a layer where each one is shown where it was detected and ,proportionately to the values.

Advantageously, the scan makes it possible to assess the length of each wrinkle.

Advantageously, the scan gives an assessment of the depth of wrinkles according to its width and the intensity of the variation of lightness.

Advantageously, the scan makes it possible to count wrinkles per zone and per category.

For skin roughness, the scanning step shall scan small cracks and/or large texture of the skin, which can be sebum excess, scars from acne, compounded loss of skin elasticity or large amount of black spots according to the age and the zones.

Advantageously, the result of the scanning step is a layer that enhances important variations in the uniformity and yields a score that can weight both the surface and the intensity of specific zones.

For skin redness conditions, the process starts first a pixel by pixel scan of an average colour of the skin that is a function of the skin complexion and the general condition of the user.

Then, in a second time, it proceeds with a scan relative to the average of the tendancy of the skin to redden, with the red selected in a collection of photographs of skin rashs and sensitive skin samples.

For example, make-up can easily hide zones with rash, but seldom divert and generate false positive results when the reference colour is identified.

Advantageously, the spotted dots are only those that draw size and shape zones of the face, which excludes wounds or buttons.

Advantageously, the scan brings out irritations or extreme dryness or a specific density resulting to a burn.

For moisture conditions, the analysis refers to a symptom of oily skins: the brightness and the ability to reflect the ambient light.

A default of moisture results in dull skin. A few dots are excluded, such as the tip of the nose and the top of the forehead in case of baldness.

A scan can be rather easily flawed by the make-up powder, while a scan before and after application of cream under controlled lighting according to the invention is very reliable.

For the radiance conditions, the analysis measures the pigmentation difference between 2 dots close to each other and repeats the process on many dots in every zones. As soon as a great surface is detected very far from the average value, its contour is systematically excluded from the measures.

Thus, the algorithm gives a global score that is a weighting between the discrepancy of the measured values and the number of dots with significant discrepancies. For example, the measurement zones exclude the lower part of the face from the tip of the nose.

For skin defaults conditions, the analysis entirely relies on machine learning. A neural network is trained to recognize buttons and zones containing emerging buttons that can be detected thanks to small bumps and a tendancy to turn red at specific locations.

For example, this development relies on a database of 2000 acne-positive pictures in a base of more than 20.000 skin defaults.

Advantageously, the more commented pictures in the picture, the more accurate the system.

Advantageously, the system automatically corrects the lighting issues.

The fourth step consists in a rating from the analysis with a weighting of both the surface and the intensity of the zones.

The algorithm gives a global score that is a weighting between the discrepancy of the measured values and the number of dots with significant discrepancies. For example, the score will take into account the age of the user and their wrinkles. This rating system is valid for each issue.

The reliability of the rating relies on an analysis on an user at a given moment in given conditions. A rating according to the age only or relative to other people is flawed.

The fifth step consists in the creation of a tailor-made cosmetic formulation.

The analysis and the rating make it possible to initiate a selection process of a tailor-made cosmetic formulation with at least 1 base and at least 1 active ingredient. To do so, at least one condition is retained to identify which active ingredient(s) the tailor-made cosmetic formulation should comprise.

The selection relies on the determination of the gravity of a condition, i.e. roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes, taking the age of the user into account. Then, the system compares the gravity of said selected condition in reference to other people of similar or equal age. Then, the system gives it a score. If this score is higher than the average, the system advantageously assigns a specific active ingredient. If this score is lower than the average, the system advantageously assigns a specific active ingredient too.

The system identifies the active ingredient(s) thanks to a matrix as a function of the age, of the selection of the condition to cure and of the gravity of each condition.

The system also identifies the base(s) according to the condition to cure to design a tailor-made cosmetic formulation.

If the base is a gel, a serum or a cream, the formulation base shall be water-based.

If the base is a balm, the formulation base shall be an aqueous base and an oil base.

If the base is an oil, the formulation base shall be an oil base.

Thus, the .system advantageously assigns the base(s) in synergy with the active ingredient(s), hence the efficiency of the tailor-made cosmetic formulation.

The process advantageously allows the design of many different formulations, i.e. at least more than 40.000 different formulations.

The sixth step consists in the creation of a tailor-made cosmetic product from a tailor-made cosmetic formulation.

According to an embodiment, all the steps of the invention can be implemented by only one system or appliance. This means an appliance capable of achieving the scanning, the various analyses, the suggestion of a formulation and the production of a tailor-made cosmetic product.

According to another embodiment, on one hand, the production of a cosmetic product can be made thanks to an appliance capable of scanning, analyzing and suggesting a formulation according to the steps 1 to 5. For example, a smart mirror (see [FIG. 2a]). On the other hand, said appliance can be linked to a device to produce a tailor-made cosmetic product.

The seventh step consists in a comparative analysis before and after application of the cosmetic product. The user will be able to assess their cosmetic product and measure the efficiency of the active ingredients that aim at the selected conditions.

The FIG. 2a shows an appliance 5, 6 according to the invention to detect the face 7 of an user.

The FIG. 2b shows an appliance according to a variant of the invention capable of producing a tailor-made cosmetic product. According to a variant of the invention, the production of a tailor-made cosmetic product (4) is achieved thanks to a production appliance of a tailor-made cosmetic formulation (1) that includes a set of bases (2) according to the invention and a set of active ingredients (3) according to the invention.

What is claimed is:

1. A process to identify a tailor-made formula adapted to an user in the cosmetic area, including:
a step of collecting picture or video information,
a step of storing said information into a database,
a step of analysis of at least one skin condition from the collected information,
a step of comparison of the collected information with the information in the database,
a step of rating from the analysis and for each skin condition,
a step of devising analysis results,
a step of submission of a cosmetic formulation from the analysis result, the cosmetic formulation including at least a cosmetic base and at least an active ingredient tailored to the user, characterized in the fact that the process includes a step of delineation of the contours of the face and a step of dividing the face in zones.

2. The process according to claim 1, characterized in that at least one active ingredient is identified for at least one of the conditions selected among roughness, cracks of the skin, sebum excess, scars from acne, compounded loss of skin elasticity, the skin grain, the wrinkles, the pigmentation, skin radiance, skin brightness, redness, sensitivity, dryness, the skin moisture and defaults like moles, spots, black spots, dark circles or puffiness under the eyes.

3. The process according to the claim 1, characterized in that a scan of the face is made on at least 70% of the contour of the face, including benchmarks selected among the eyes, the nostrils or the lips.

4. The process according to the claim 3, characterized in that the scan on a full face is at least 99.8%.

5. The process according to the claim 3, characterized in that the scan on a full face with a marked zone of the skin is at least 98%.

6. The process according to the claim 1, characterized in that a cosmetic base is selected among a cream, a serum, an oil, a balm, a gel, a creamy gel, an emulsion or a combination of the same.

7. The process according claim 1, characterized in that figuring out a tailor-made cosmetic formulation includes the following steps:
a step of taking a photograph and detecting the face in the photograph;
a step of delineation of the contours of the face for analysis and division of the face in zones;
a step of analysis of the skin from the photograph;
a step of rating from the analysis;
a step of creation of a tailor-made cosmetic formulation;
a step of creation of a tailor-made cosmetic product from a tailor-made cosmetic formulation;
a step of comparative analysis before and after application of the cosmetic product.

8. A system capable to implement the process according to claim 1, including a device for scanning, analysis, suggestion and manufacture of a tailor-made cosmetic formulation, wherein the system is of the type of a mirror.
